(19) United States
(12) Patent Application Publication
(10) Pub. No.: US 2014/0371317 A1
(43) Pub. Date: Dec. 18, 2014

(54) Silicone Resin Emulsions

(71) Applicant: Dow Corning Corporation, MIDLAND, MI (US)

(72) Inventors: Hyder Aliyar, Midland, MI (US); Robert O. Huber, Midland, MI (US); Donald Taylor Liles, Midland, MI (US); Gary L. Loubert, Saginaw, MI (US); Gerald K. Schalau, II, Freeland, MI (US); SIMON Toth, Midland, MI (US)

(73) Assignee: Dow Corning Corporation, MIDLAND, MI (US)

(21) Appl. No.: 14/372,276

(22) PCT Filed: Feb. 6, 2013
(86) PCT No.: PCT/US13/24897

§ 371 Date: Jul. 15, 2014

Related U.S. Application Data
(60) Provisional application No. 61/596,328, filed on Feb. 8, 2012.
(60) Provisional application No. 61/596,342, filed on Feb. 8, 2012.
(60) Provisional application No. 61/596,340, filed on Feb. 8, 2012.

(51) Int. Cl. A61K 47/24; A61K 47/34; A61K 9/107; A61K 47/02; A61K 31/192; A61K 31/196

(52) U.S. Cl. CPC A61K 47/24; A61K 31/192; A61K 31/196; A61K 9/107; A61K 47/02; A61K 47/34
USPC 514/567; 514/570

(57) ABSTRACT

The present disclosure relates to compositions comprising: i) an aqueous silicone emulsion comprising; A) 0.5 wt % to 95 wt % of a silicone gum, resin, or PSA, B) 0.1 to 90 wt % of an ethylene oxide/propylene oxide block copolymer, and sufficient amount of water to sum all ingredients of the silicone gum emulsion to 100 weight percent, ii) a healthcare active, and iii) an optional enhancer(s).

### CROSS-REFERENCE TO RELATED APPLICATIONS

This application claims the benefit of U.S. Application No. 61/596,328 as filed on 8 Feb. 2012, U.S. Application No. 61/596,340 as filed on 8 Feb. 2012, and U.S. Application No. 61/596,342 as filed on 8 Feb. 2012. U.S. Application Nos. 61/596,328, 61/596,340, and 61/596,342 are herein incorporated by reference in their entirety.

### BACKGROUND OF THE INVENTION

Dermal formulations carry an active healthcare or pharmaceutical ingredient across the skin barrier while also satisfying sensorial properties to drive patient compliance and product differentiation. As such, there is a continual need to identify improved dermal formulations that are cost efficient, easy to manufacture, and provide sufficient formulation latitude to accommodate a range of various hydrophobic and hydrophilic drugs.

### BRIEF SUMMARY OF THE INVENTION

The present inventors have found certain silicone gum, resin, or pressure sensitive adhesive (PSA) emulsions may be used to prepare compositions for dermal formulations to deliver healthcare actives.

The present disclosure relates to compositions comprising:

i) an aqueous silicone gum emulsion comprising;
A) 0.5 wt % to 95 wt % of a silicone gum, resin, PSA,
B) 0.1 to 90 wt % of an ethylene oxide/propylene oxide block copolymer,
and sufficient amount of water to sum all ingredients of the silicone gum emulsion to 100 weight percent,

ii) a healthcare active, and

iii) an optional enhancer(s).

### BRIEF SUMMARY OF THE DRAWINGS

FIG. 1 Flux profile for formulation examples 1-3 and bench mark

FIG. 2 Flux profile for formulation examples 4-6 and bench mark

FIG. 3 Flux profile for formulation examples 7-9 and bench mark

FIG. 4 Flux profile for formulation examples 10-12 and bench mark

### DETAILED DESCRIPTION OF THE INVENTION

### i) The Silicone Emulsion

The present compositions comprise a silicone emulsion as component i). The silicone emulsion comprises;
A) 0.5 wt % to 95 wt % of a silicone gum, resin, or PSA.
B) 0.1 to 90 wt % of an ethylene oxide/propylene oxide block copolymer,
and sufficient amount of water to sum all ingredients of the silicone gum emulsion to 100 weight percent.

Component A) in the silicone emulsions used in the present compositions may be selected from a silicone gum, silicone resin, or silicone pressure sensitive adhesive composition. “Silicone gum” as used herein refers to predominately linear organopolysiloxanes having sufficiently high molecular weight (Mw) to provide kinetic viscosities greater than 500 thousand cSt at 25° C. As used herein, “silicone resin” refers to any organopolysiloxane containing at least one (RSiO_{3/2}), or (SiO_{4/2}) siloxy unit. As used herein in its broadest sense, a silicone PSA refers to the reaction products resulting from reacting a hydroxyl endblocked “linear” organopolysiloxane with a “resin” organopolysiloxane, wherein the resin organopolysiloxane contains at least one (RSiO_{3/2}), or (SiO_{4/2}) siloxy unit.

Organopolysiloxanes are polymers containing siloxy units independently selected from (R₃SiO_{1/2}), (R₂SiO_{2/2}), (RSiO_{3/2}), or (SiO_{4/2}) siloxy units, where R may be any organic group. These siloxy units are commonly referred to as M, D, T, and Q units respectively. These siloxy units can be combined in various manners to form cyclic, linear, or branched structures. The chemical and physical properties of the resulting polymeric structures vary depending on the number and type of siloxy units in the organopolysiloxane. “Linear” organopolysiloxanes typically contain mostly D or (R₂SiO_{2/2}) siloxy units, which results in polydiorganosiloxanes that are fluids of varying viscosity, depending on the “degree of polymerization” or DP as indicated by the number of D units in the polydiorganosiloxane. “Linear” organopolysiloxanes typically have glass transition temperatures (T_{g}) that are lower than 25° C. “Resin” organopolysiloxanes result when a majority of the siloxy units are selected from T or Q siloxy units. When T siloxy units are predominately used to prepare an organopolysiloxane, the resulting organosiloxane is often referred to as a “silsesquioxane resin”. When M and Q siloxy units are predominately used to prepare an organopolysiloxane, the resulting organosiloxane is often referred to as a “MQ resin”. Alternatively, the formula for an organopolysiloxane may be designated by the average of the siloxy units in the organopolysiloxane as follows; RₙSiO_{(4-n)/2}, where the R is independently any organic group, alternatively a hydrocarbon, or alternatively an alkyl group, or alternatively methyl. The value of n in the average formula may be used to characterize the organopolysiloxane. For example, an average value of n=1 would indicate a predominate concentration of the (RSiO_{3/2}) siloxy unit in the organopolysiloxane, while n=2 would indicate a predominance of (R₂SiO_{2/2}) siloxy units. As used herein, “organopolysiloxane resin” refers to those organopolysiloxanes having a value of n less than 1.8 in the average formula RₙSiO_{(4-n)/2}, indicating a resin.

Suitable silicone gum, resin, and PSA compositions useful as component A) are further described as follows.

Component A) may be a silicone gum. “Silicone gum” as used herein refers to predominately linear organopolysiloxanes having sufficiently high molecular weight (Mw) to provide kinetic viscosities greater than 500 thousand cSt at 25° C. While any organopolysiloxane considered as a gum may be selected as component (A), typically the silicone gum is a diorganopolysiloxane gum with a molecular weight sufficient to impart a William's plasticity number of at least about 30 as determined by the American Society for Testing and Materials (ASTM) test method 926. The silicon-bonded organic groups of the diorganopolysiloxane may independently be selected from hydrocarbon or halogenated hydrocarbon groups. These may be specifically exemplified by alkyl groups having 1 to 20 carbon atoms, such as methyl, ethyl, propyl, butyl, pentyl and hexyl; cycloalkyl groups, such as cyclohexyl and cycloheptyl; aryl groups having 6 to 12 carbon atoms, such as phenyl, tolyl and xylyl; aralkyl groups having 7 to 20 carbon atoms, such as benzyl and phenylethyl; and halogenated alkyl groups having 1 to 20 carbon atoms, such as 3,3,3-trifluoropropyl and chloromethyl. Thus, diorganopolysiloxane can be a homopolymer, a copolymer or a terpolymer containing such organic groups. Examples include homopolymers comprising dimethylsiloxy units, homopolymers comprising 3,3,3-trifluoropropylmethylsiloxy units, copolymers comprising dimethylsiloxy units and phenylmethylsiloxy units, copolymers comprising dimethylsiloxy units and 3,3,3-trifluoropropylmethylsiloxy units, copolymers of dimethylsiloxy units and diphenylsiloxy units and interpolymers of dimethylsiloxy units, diphenylsiloxy units and phenylmethylsiloxy units, among others.

The silicon-bonded organic groups of the diorganopolysiloxane may also be selected from alkenyl groups having 1 to 20 carbon atoms, such as vinyl, allyl, butyl, pentyl, hexenyl, or dodecenyl. Examples include; dimethylvinylsiloxy-endblocked dimethylpolysiloxanes; dimethylvinylsiloxy-endblocked dimethylsiloxane-methylvinylsiloxane copolymers; dimethylvinylsiloxy-endblocked methylphenylpolysiloxanes; dimethylvinylsiloxy-endblocked methylphenylsiloxane-dimethylsiloxane-methylvinylsiloxane copolymers.

The silicon-bonded organic groups of the diorganopolysiloxane may also be selected from various organofunctional groups such as amino, amido, mercapto, or epoxy functional groups.

The molecular structure is also not critical and is exemplified by straight-chain and partially branched straight-chain structures, the linear systems being the most typical.

The silicone gum used as component A) may also be a combination or mixture of any of the aforementioned polydiorganosiloxanes.

In one embodiment, the silicone gum is a hydroxy terminated polydimethylsiloxane gum having a viscosity of at least 20 million cP at 25° C. at 0.01 Hz.

The silicone gum may be used in combination with other organopolysiloxanes.

The amount of the additional organopolysiloxane combined with the silicone gum may vary. Typically, 0.1 parts to 1000 parts by weight, alternatively 0.1 to 100 parts by weight of the additional organopolysiloxane is added for every 100 parts of the silicone gum.

The silicone resin useful as component A) may independently comprise (i) (R¹₃SiO_{1/2})ₐ, (ii) (R²₂SiO_{2/2})_{b}, (iii) (R³SiO_{3/2})_{c}, and (iv) (SiO_{4/2})_{d} siloxy units, providing there is at least one T or Q siloxy unit in the silicone resin molecule. The amount of each unit present in the silicone resin is expressed as a mole fraction (i.e. a, b, c, or d) of the total number of moles of all M, D, T, and Q units present in the silicone resin. Any such formula used herein to represent the silicone resin does not indicate structural ordering of the various siloxy units. Rather, such formulae are meant to provide a convenient notation to describe the relative amounts of the siloxy units in the silicone resin, as per the mole fractions described above via the subscripts a, b, c, and d. The mole fractions of the various siloxy units in the present organosiloxane block copolymers, as well as the silanol content, may be readily determined by ²⁹Si NMR techniques.

The silicone resin may also contain silanol groups (SiOH). The amount of silanol groups present on the silicone resin may vary from 0.1 to 35 mole percent silanol groups [═SiOH], alternatively from 2 to 30 mole percent silanol groups [═SiOH], alternatively from 5 to 20 mole percent silanol groups [═SiOH]. The silanol groups may be present on any siloxy units within the silicone resin.

The molecular weight of the silicone resin is not limiting. The silicone resin may have an average molecular weight (M_{W}) of at least 1,000 g/mole, alternatively an average molecular weight of at least 2,000 g/mole alternatively an average molecular weight of at least 5,000 g/mole. The average molecular weight may be readily determined using Gel Permeation Chromatography (GPC) techniques.

In one embodiment, the silicone resin is a MQ silicone. The silicone resin may be a MQ resin comprising at least 80 mole % of siloxy units selected from (R¹₃SiO_{1/2})ₐ and (SiO_{4/2})_{d} units (that is a+d≧0.8), where R¹ is an alkyl group having from 1 to 8 carbon atoms, an aryl group, a carbinol group, or an amino group, with the proviso that at least 95 mole % of the R¹ groups are alkyl groups, a and d each have a value greater than zero, and the ratio of a/d is 0.5 to 1.5.

The R¹ units of the MQ resin are independently an alkyl group having from 1 to 8 carbon atoms, an aryl group, a carbinol group, or an amino group. The alkyl groups are illustrated by methyl, ethyl, propyl, butyl, pentyl, hexyl, and octyl. The aryl groups are illustrated by phenyl, naphthyl, benzyl, tolyl, xylyl, xenyl, methylphenyl, 2-phenylethyl, 2-phenyl-2-methylethyl, chlorophenyl, bromophenyl and fluorophenyl with the aryl group typically being phenyl.

MQ resins suitable for use as component (A), and methods for their preparation, are known in the art. For example, U.S. Pat. No. 2,814,601 to Currie et al., Nov. 26, 1957, which is hereby incorporated by reference, discloses that MQ resins can be prepared by converting a water-soluble silicate into a silicic acid monomer or silicic acid oligomer using an acid. When adequate polymerization has been achieved, the resin is end-capped with trimethylchlorosilane to yield the MQ resin. Another method for preparing MQ resins is disclosed in U.S. Pat. No. 2,857,356 to Goodwin, Oct. 21, 1958, which is hereby incorporated by reference. Goodwin discloses a method for the preparation of an MQ resin by the cohydrolysis of a mixture of an alkyl silicate and a hydrolyzable trialkylsilane organopolysiloxane with water.

The MQ resins suitable as component A) in the present invention may contain D and T units. The MQ resins may also contain hydroxy groups. Typically, the MQ resins have a total weight % hydroxy content of 2-10 weight %, alternatively 2-5 weight %. The MQ resins can also be further “capped” wherein residual hydroxy groups are reacted with additional M groups.

In one embodiment, the silicone resin is a silsesquioxane resin. The silsesquioxane resin may be a silsesquioxane resin comprising at least 80 mole % of R³SiO_{3/2} units, where R³ in the above trisiloxy unit formula is independently a C₁ to C₂₀ hydrocarbyl, a carbinol group, or an amino group. As used herein, hydrocarbyl also includes halogen substituted hydrocarbyls. R³ may be an aryl group, such as phenyl, naphthyl, anthryl group. Alternatively, R³ may be an alkyl group, such as methyl, ethyl, propyl, or butyl. Alternatively, R³ may be any combination of the aforementioned alkyl or aryl groups. Alternatively, R³ is phenyl, propyl, or methyl. In one embodiment, at least 40 mole % of the R³ groups are propyl, referred herein as T-propyl resins, since the majority of the siloxane units are T units of the general formula R³SiO_{3/2} where at least 40 mole %, alternatively 50 mole %, or alternatively 90 mole % of the R³ groups are propyl. In another embodiment, at least 40 mole % of the R³ groups are phenyl, referred herein as T-phenyl resins, since the majority of the siloxane units are T units of the general formula R³SiO_{3/2} where at least 40 mole %, alternatively 50 mole %, or alternatively 90 mole % of the R³ groups are phenyl. In yet another embodiment, R³ may be a mixture of propyl and phenyl. When R³ is a mixture of propyl and phenyl, the amounts of each in the resin may vary, but typically the R³ groups in the silsesquioxane resin may contain 60-80 mole percent phenyl and 20-40 mole percent propyl.

Silsesquioxane resins are known in the art and are typically prepared by hydrolyzing an organosilane having three hydrolyzable groups on the silicon atom, such as a halogen or alkoxy group. Thus, silsesquioxane resins can be obtained by hydrolyzing propyltrimethoxysilane, propyltriethoxysilane, propyltripropoxysilane, or by co-hydrolyzing the aforementioned propylalkoxysilanes with various alkoxysilanes. Examples of these alkoxysilanes include methyltrimethoxysilane, methyltriethoxysilane, methyltriisopropoxysilane, dimethyldimethoxysilane, and phenyltrimethoxysilane. Propyltrichlorosilane can also be hydrolyzed alone, or in the presence of alcohol. In this case, co-hydrolyzation can be carried out by adding methyltrichlorosilane, dimethyldichlorosilane, phenyltrichlorosilane, or similar chlorosilanes and methyltrimethoxysilane, methyltriethoxysilane, methyltriisopropoxysilane, or similar methylalkoxysilane. Alcohols suitable for these purposes include methanol, ethanol, n-propyl alcohol, isopropyl alcohol, butanol, methoxy ethanol, ethoxy ethanol, or similar alcohols. Examples of hydrocarbon-type solvents which can also be concurrently used include toluene, xylene, or similar aromatic hydrocarbons; hexane, heptane, isooctane, or similar linear or partially branched saturated hydrocarbons; and cyclohexane, or similar aliphatic hydrocarbons.

The silsesquioxane resins suitable in the present disclosure may contain M, D, and Q units, but typically at least 80 mole %, alternatively 90 mole % of the total siloxane units are T units. The silsesquioxane resins may also contain hydroxy and/or alkoxy groups. Typically, the silsesquioxane resins have a total weight % hydroxy content of 2-10 weight % and a total weight % alkoxy content of up to 20 weight %, alternatively 6-8 weight % hydroxy content and up to 10 weight % alkoxy content.

Representative, non-limiting examples of commercial silicone resins suitable as component A) include; silicone resins sold under the trademarks DOW CORNING® 840 Resin, DOW CORNING® 2-7466 Resin, DOW CORNING® 2-9138 Resin, DOW CORNING® 2-9148 Resin, DOW CORNING® 2104 Resin, DOW CORNING® 2106 Resin, DOW CORNING® 217 Flake Resin, DOW CORNING® 220 Flake Resin, DOW CORNING® 233 Flake Resin, DOW CORNING® 4-2136 Resin, Xiameter® RSN-6018 Resin, Xiameter® RSN-0217 Resin, Silres® MK methyl silicone resin, Dow Corning® MQ 1600 Resin.

As used herein, “silicone resin” also encompasses silicone-organic resins. Thus, silicone-organic resins includes silicone-organic copolymers, where the silicone portion contains at least one (RSiO_{3/2}), or (SiO_{4/2}) siloxy unit. The silicone portion of the silicone-organic resin may be any of the silisesquioxane or MQ resins as described above. The organic portion may be any organic polymer, such as those derived by free radical polymerization of one or more ethylenically unsaturated organic monomers. Various types of ethylenically unsaturated and/or vinyl containing organic monomers can be used to prepare the organic portion including; acrylates, methacrylates, substituted acrylates, substituted methacrylates, vinyl halides, fluorinated acrylates, and fluorinated methacrylates, for example. Some representative compositions include acrylate esters and methacrylate esters such as methyl acrylate, ethyl acrylate, butyl acrylate, 2-ethylhexyl acrylate, methyl methacrylate, decyl acrylate, lauryl acrylate, isodecyl methacrylate, lauryl methacrylate, and butyl methacrylate; substituted acrylates and methacrylates such as hydroxyethyl acrylate, perfluorooctyl acrylate, hydroxypropyl acrylate, hydroxypropyl methacrylate, and hydroxyethyl methacrylate; vinyl halides such as vinyl chloride, vinylidene chloride, and chloroprene; vinyl esters such as vinyl acetate and vinyl butyrate; vinyl pyrrolidone; conjugated dienes such as butadiene and isoprene; vinyl aromatic compounds such as styrene and divinyl benzene; vinyl monomers such as ethylene; acrylonitrile and methacrylonitrile; acrylamide, methacrylamide, and N-methylol acrylamide; and vinyl esters of monocarboxylic acids

The silicone resin selected as component A) may also be a combination(s) of any of the aforementioned silicone resins.

When component A) is a silicone PSA, it may be the reaction product of a hydroxy endblocked polydimethylsiloxane polymer and a hydroxy functional silicate or silicone resin. Typically, the hydroxy functional silicate resin is a trimethylsiloxy and hydroxy endblocked silicate resin, such as the silicone resins described above. The polydimethylsiloxane polymer and hydroxy functional silicate resin are reacted in a condensation reaction to form the silicone PSA.

PSAs are disclosed in U.S. Pat. Nos. 4,584,355; 4,585,836; 4,591,622; 5,726,256; 5,776,614; 5,861,472; 5,869,556; 6,337086, all of which are hereby incorporated by reference for the purpose of disclosing the chemical compositions of PSAs useful as component A) in the present disclosure.

The silicone PSA may also be a silicone acrylate hybrid composition, as disclosed in WO2007/145996, which is incorporated herein by reference for its teaching of suitable PSA compositions as component A).

Representative, non-limiting examples of commercially available PSA's suitable as component A) include; Dow Corning@ Q2-7406 Adhesive, Dow Corning® Q2-7735 Adhesive, Dow Corning@ 7355 Adhesive, Dow Corning® 7358 Adhesive, Dow Corning® Q2-7566 Adhesive, Dow Corning® 7-4100 Adhesive, Dow Corning® 7-4200 Adhesive, Dow Corning® 7-4300 Adhesive, Dow Corning® 7-4400 Adhesive, Dow Corning® 7-4500 Adhesive, Dow Corning@ 7-4600 Adhesive, Dow Corning® 7-4560, Shin-Etsu KR-100, Shin-Etsu KR-101-10, Shin-Etsu SR-130 Momentive PSA518, Momentive SPUR+PSA 3.0, Momentive SILGRIP PSA529, Momentive SILGRIP PSA915, Momentive SILGRIP PSA610, Momentive SILGRIP PSA595, Momentive SILGRIP PSA6374, and Momentive SILGRIP PSA6574.

### B) The Ethylene Oxide/Propylene Oxide Block Copolymer

Component B) is an ethylene oxide/propylene oxide block copolymer. Component B) may be selected from those ethylene oxide/propylene oxide block copolymers known to have surfactant behavior. Typically, the ethylene oxide/propylene oxide block copolymers useful as component B) are surfactants having an HLB of at least 12, alternatively, at least 15, or alternatively at least 18.

The molecular weight of the ethylene oxide/propylene oxide block copolymer may vary, but typically is at least 4,000 g/mol, alternatively at least 8,000 g/mol, or at least 12,000 g/mol.

The amounts of ethylene oxide (EO) and propylene oxide (PO) present in the ethylene oxide/propylene oxide block copolymer may vary, but typically, the amount of EO may vary from 50 percent to 80 percent, or alternatively from 60 percent to about 85 percent, or alternatively from 70 percent to 90 percent.

In one embodiment, component B) is a poly(oxyethylene)-poly(oxypropylene)-poly(oxyethylene) tri-block copolymer. Poly(oxyethylene)-poly(oxypropylene)-poly(oxyethylene) tri-block copolymers are also commonly known as Poloxamers. They are nonionic triblock copolymers composed of a central hydrophobic chain of polyoxypropylene (poly(propylene oxide)) flanked by two hydrophilic chains of polyoxyethylene (poly(ethylene oxide)).

Poly(oxyethylene)-poly(oxypropylene)-poly(oxyethylene) tri-block copolymers are commercially available from BASF (Florham Park, N.J.) and are sold under the tradename PLURONIC®. Representative, non-limiting examples suitable as component (B) include; PLURONIC® F127, PLURONIC® F98, PLURONIC® F88, PLURONIC® F87, PLURONIC® F77 and PLURONIC® F68, and PLURONIC® F-108.

In a further embodiment, the poly(oxyethylene)-poly(oxypropylene)-poly(oxyethylene) tri-block copolymer has the formula;

HO(CH₂CH₂O)ₘ(CH₂CH(CH₃)O)ₙ(CH₂CH₂O)ₘH
where the subscript “m” may vary from 50 to 400, or
   alternatively from 100 to 300,
and the subscript “n” may vary from 20 to 100, or
   alternatively from 25 to 100.

In one embodiment, component B) is a tetrafunctional poly(oxyethylene)-poly(oxypropylene) block copolymer derived from the sequential addition of propylene oxide and ethylene oxide to ethylene diamine. These tetra-functional block copolymers are also commonly known as Poloxamines. The tetrafunctional poly(oxyethylene)-poly(oxypropylene) block copolymer may have the average formula;

[HO(CH₂CH₂O)_{q}(CH₂CH(CH₃)O)ᵣ]₂NCH₂CH₂N[(CH₂CH(CH₃)O)ᵣ(CH₂CH₂O)_{q}H]₂
where the subscript “q” may vary from 50 to 400, or
   alternatively from 100 to 300,
and the subscript “r” may vary from 15 to 75, or
   alternatively from 20 to 50.

Tetrafunctional poly(oxyethylene)-poly(oxypropylene) block copolymers are commercially available from BASF (Florham Park, N.J.) and are sold under the tradename TETRONIC®. Representative, non-limiting examples suitable as component (B) include; TETRONIC® 908, TETRONIC® 1107, TETRONIC® 1307, TETRONIC® 1508 and TETRONIC® 1504.

The amounts of components A) and B) may vary in the emulsion. Typically the silicone emulsions comprise, alternatively consists essentially of, or alternatively consists of:

0.5 to 95 wt. % of A) the silicone gum, resin, or PSA;
alternatively 5 to 90 wt. % of A) silicone gum, resin, or PSA,
alternatively 10 to 80 wt. % of A) silicone gum, resin, or PSA,
alternatively 20 to 70 wt. % of A) silicone gum, resin, or PSA,
alternatively 30 to 60 wt. % of A) silicone gum, resin, or PSA,

0.1 to 90 wt. % of B) the ethylene oxide/propylene oxide block copolymer;
alternatively 0.1 to 50 wt. % of B) the block copolymer,
   alternatively 0.5 to 40 wt. % of B) the block copolymer,
   alternatively 1 to 30 wt. % of B) the block copolymer,
      alternatively 1 to 20 wt. % of B) the block copolymer,
      alternatively 1 to 10 wt. % of B) the block copolymer,
         and sufficient amounts of water, or other components, to sum to 100 wt %.

Other additives can also be incorporated in the emulsions of the present disclosure, such as fillers, preservatives, biocides, freeze/thaw additives, anti-freeze agents, various thickeners, viscosity modifiers, and foam control agents.

The emulsion compositions of the present disclosure may be an oil/water emulsion, a water/oil emulsion, a multiple phase or triple emulsion.

In one embodiment, the emulsion products produced by the present process are “oil/water emulsions”, that is, an emulsion having an aqueous continuous phase and a dispersed phase comprising the silicone gum resin, or PSA. The oil/water emulsion may be characterized by average volume particle of the dispersed silicone (oil) phase in a continuous aqueous phase. The particle size may be determined by laser diffraction of the emulsion. Suitable laser diffraction techniques are well known in the art. The particle size is obtained from a particle size distribution (PSD). The PSD can be determined on a volume, surface, length basis. The volume particle size is equal to the diameter of the sphere that has the same volume as a given particle. The term Dv represents the average volume particle size of the dispersed particles. Dv 50 is the particle size measured in volume corresponding to 50% of the cumulative particle population. In other words if Dv 50=10 μm, 50% of the particle have an average volume particle size below 10 μm and 50% of the particle have a volume average particle size above 10 μm. Dv 90 is the particle size measured in volume corresponding to 90% of the cumulative particle population.

The average volume particle size of the dispersed silicone particles in the oil/water emulsions is between 0.1 μm and 150 μm; or between 0.1 μm and 30 μm; or between 0.3 μm and 5.0 μm.

The present emulsions may be prepared by any known methods, or alternatively prepared by the methods as discussed below.

The silicone emulsions may be prepared by;
I) forming a dispersion of;
   A) 100 parts of a silicone gum, resin, or PSA
   B) 5 to 100 parts of a ethylene oxide/propylene oxide block copolymer,
II) admixing a sufficient amount of water to the dispersion from step I) to form an emulsion,
III) optionally, further shear mixing the emulsion.

The amount of components A) and B) combined in step I) are as follows;

A) 100 parts of a silicone gum, resin, or PSA, and

B) 5 to 100 parts, alternatively 10 to 40 parts, or alternatively 10 to 25 of the ethylene oxide/propylene oxide block copolymer. Components A) and B) are the same as described above.

As used herein, “parts” refers to parts by weight.

In one embodiment, the dispersion formed in step I) consists essentially of components A) and B) as described above. In this embodiment, no additional surfactants or emulsifiers are added in step I). Furthermore, no solvents are added for the purpose of enhancing formation of an emulsion. As used herein, the phrase “essentially free of “solvents” means that solvents are not added to components A) and B) in order to create a mixture of suitable viscosity that can be processed on typical emulsification devices. More specifically, “solvents” as used herein is meant to include any water immiscible low molecular weight organic or silicone material added to the non-aqueous phase of an emulsion for the purpose of enhancing the formation of the emulsion, and is subsequently removed after the formation of the emulsion, such as evaporation during a drying or film formation step. Thus, the phrase “essentially free of solvent” is not meant to exclude the presence of solvent in minor quantities in process or emulsions of the present invention. For example, there may be instances where the components A) and B) may contain minor amounts of solvent as supplied commercially. Small amounts of solvent may also be present from residual cleaning operations in an industrial process. Preferably, the amount of solvent present in the premix should be less than 2% by weight of the mixture, and most preferably the amount of solvent should be less than 1% by weight of the mixture.

The dispersion of step (I) may be prepared by combining components A) and B) and further mixing the components to form a dispersion. The resulting dispersion may be considered as a homogenous mixture of the two components. The present inventors have unexpectedly found that certain ethylene oxide/propylene oxide block copolymers readily disperse with silicone gum compositions, and hence enhance the subsequent formation of emulsion compositions thereof. The present inventors believe other nonionic and/or anionic surfactants, typically known for preparing silicone emulsions, do not form such dispersions or homogeneous mixtures upon mixing with a silicone gum, resin, or PSA (at least not in the absence of a solvent or other substance to act as a dispersing medium). While not wishing to be limited to any theory, the inventors believe the discovery of the present ethylene oxide/propylene oxide block copolymers to form such dispersions with silicone gums, resins, and PSAs provides emulsion compositions of these silicones without the presence of undesirable solvents, or requiring elaborate handling/mixing techniques.

Mixing can be accomplished by any method known in the art to effect mixing of high viscosity materials. The mixing may occur either as a batch, semi-continuous, or continuous process. Mixing may occur, for example using, batch mixing equipments with medium/low shear include change-can mixers, double-planetary mixers, conical-screw mixers, ribbon blenders, double-arm or sigma-blade mixers; batch equipments with high-shear and high-speed dispersers include those made by Charles Ross & Sons (NY), Hockmeyer Equipment Corp. (NJ); batch mixing equipment such as those sold under the tradename Speedmixer®; batch equipments with high shear actions include Banbury-type (CW Brabender Instruments Inc., NJ) and Henschel type (Henschel mixers America, Tex.). Illustrative examples of continuous mixers/compounders include extruders single-screw, twin-screw, and multi-screw extruders, co-rotating extruders, such as those manufactured by Krupp Werner & Pfleiderer Corp (Ramsey, N.J.), and Leistritz (NJ); twin-screw counter-rotating extruders, two-stage extruders, twin-rotor continuous mixers, dynamic or static mixers or combinations of these equipments.

The process of combining and mixing components A) and B) may occur in a single step or multiple step process. Thus, components A) and B) may be combined in total, and subsequently mixed via any of the techniques described above. Alternatively, a portion(s) of components A) and B) may first be combined, mixed, and followed by combining additional quantities of either or both components and further mixing. One skilled in the art would be able to select optimal portions of components A) and B) for combing and mixing, depending on the selection of the quantity used and the specific mixing techniques utilized to perform step I) to provide a dispersion of components A) and B).

Step II of the process involves admixing sufficient water to the mixture of step Ito form an emulsion. Typically 5 to 700 parts water are mixed for every 100 parts of the step I mixture to form an emulsion. In one embodiment the emulsion formed is a water continuous emulsion. Typically, the water continuous emulsion has dispersed particles of the silicone from step I, and having an average particle size less than 150 μm.

The amount of water added in step II) can vary from 5 to 700 parts per 100 parts by weight of the mixture from step I. The water is added to the mixture from step I at such a rate so as to form an emulsion of the mixture of step I. While this amount of water can vary depending on the selection of the amount of silicone gum present and the specific ethylene oxide/propylene oxide block copolymer used, generally the amount of water is from 5 to 700 parts per 100 parts by weight of the step I mixture, alternatively from 5 to 100 parts per 100 parts by weight of the step I mixture, or alternatively from 5 to 70 parts per 100 parts by weight of the step I mixture.

Typically the water is added to the mixture from step I in incremental portions, whereby each incremental portion comprises less than 30 weight % of the mixture from step I and each incremental portion of water is added successively to the previous after the dispersion of the previous incremental portion of water, wherein sufficient incremental portions of water are added to form an emulsion.

Alternatively, a portion or all the water used in step I) may be substituted with various hydrophilic solvents that are soluble with water such as low molecular weight alcohols, ethers, esters or glycols. Representative non-limiting examples include low molecular weight alcohols such as methanol, ethanol, propanol, isopropanol and the like; low molecular weight ethers such as di(propyleneglycol) mono methyl ether, di(ethyleneglycol) butyl ether, di(ethyleneglycol) methyl ether, di(propyleneglycol) butyl ether, di(propyleneglycol) methyl ether acetate, di(propyleneglycol) propyl ether, ethylene glycol phenyl ether, propylene glycol butyl ether, 1-methoxy-2-propanol, 1-methoxy-2-propyl acetate, propylene glycol propyl ether, 1-phenoxy-2-propanol, tri(propyleneglycol) methyl ether and tri(propyleneglycol) butyl ether, and other like glycols.

Mixing in step (II) can be accomplished by any method known in the art to affect mixing of high viscosity materials. The mixing may occur either as a batch, semi-continuous, or continuous process. Any of the mixing methods as described for step (I), may be used to affect mixing in step (II). Typically, the same equipment is used to effect mixing in steps I) and II).

Optionally, the water continuous emulsion formed in step (II) may be further sheared according to step (III) to reduce particle size and/or improve long term storage stability. The shearing may occur by any of the mixing techniques discussed above.

### ii) The Healthcare Active

The present compositions comprise as component ii) a healthcare active. The present compositions may contain 0.001 to 200 parts by weight of ii) the healthcare active for every 100 parts by weight of component i), the silicone gum emulsion as described above. Component ii) may be selected from the various healthcare actives subsequently listed herein below. In one embodiment, the healthcare active is a non-steroidal anti-inflammatory drug (herein NSAIDs) selected from acetyl salicylic acid, ibuprofen, naproxen, benoxaprofen, flurbiprofen, fenoprofen, fenbufen, ketoprofen, indoprofen, pirprofen, carprofen, oxaprozin, pranoprofen, mniroprofen, tioxaprofen, suprofen, alminoprofen, tiaprofenic acid, fluprofen, diclofenac, and bucloxic acid. Alternatively, the healthcare active is ibuprofen or diclofenac.

### The Health Care Active

A healthcare active is added to the present compositions. A “healthcare active” means any compound or mixtures of compounds that are known in the art to provide a pharmaceutical or medical benefit. Thus, “healthcare active” include materials consider as an active ingredient or active drug ingredient as generally used and defined by the United States Department of Health & Human Services Food and Drug Administration, contained in Title 21, Chapter I, of the Code of Federal Regulations, Parts 200-299 and Parts 300-499.

Useful active ingredients for use in the present compositions include vitamins and its derivatives, including “pro-vitamins”. Vitamins useful herein include, but are not limited to, Vitamin A1, retinol, C2-C18 esters of retinol, vitamin E, tocopherol, esters of vitamin E, and mixtures thereof. Retinol includes trans-retinol, 1,3-cis-retinol, 11-cis-retinol, 9-cis-retinol, and 3,4-didehydro-retinol, Vitamin C and its derivatives, Vitamin B 1, Vitamin B2, Pro Vitamin B5, panthenol, Vitamin B6, Vitamin B12, niacin, folic acid, biotin, and pantothenic acid. Other suitable vitamins and the INCI names for the vitamins considered included herein are ascorbyl dipalmitate, ascorbyl methylsilanol pectinate, ascorbyl palmitate, ascorbyl stearate, ascorbyl glucocide, sodium ascorbyl phosphate, sodium ascorbate, disodium ascorbyl sulfate, potassium (ascorbyl/tocopheryl) phosphate.

RETINOL, it should be noted, is an International Nomenclature Cosmetic Ingredient Name (INCI) designated by The Cosmetic, Toiletry, and Fragrance Association (CTFA), Washington D.C., for vitamin A. Other suitable vitamins and the INCI names for the vitamins considered included herein are RETINYL ACETATE, RETINYL PALMITATE, RETINYL PROPIONATE, α-TOCOPHEROL, TOCOPHERSOLAN, TOCOPHERYL ACETATE, TOCOPHERYL LINOLEATE, TOCOPHERYL NICOTINATE, and TOCOPHERYL SUCCINATE.

Some examples of commercially available products suitable for use herein are Vitamin A Acetate and Vitamin C, both products of Fluka Chemie AG, Buchs, Switzerland; COVI-OX T-50, a vitamin E product of Henkel Corporation, La Grange, Ill.; COVI-OX T-70, another vitamin E product of Henkel Corporation, La Grange, Ill.; and vitamin E Acetate, a product of Roche Vitamins & Fine Chemicals, Nutley, N.J.

The active can be a protein, such as an enzyme. The internal inclusion of enzymes in these compositions have advantages to prevent enzymes from deactivating and maintain bioactive effects of enzymes for longer time. Enzymes include, but are not limited to, commercially available types, improved types, recombinant types, wild types, variants not found in nature, and mixtures thereof. For example, suitable enzymes include hydrolases, cutinases, oxidases, transferases, reductases, hemicellulases, esterases, isomerases, pectinases, lactases, peroxidases, laccases, catalases, and mixtures thereof. Hydrolases include, but are not limited to, proteases (bacterial, fungal, acid, neutral or alkaline), amylases (alpha or beta), lipases, mannanases, cellulases, collagenases, lisozymes, superoxide dismutase, catalase, and mixtures thereof. Said protease include, but are not limited to, trypsin, chymotrypsin, pepsin, pancreatin and other mammalian enzymes; papain, bromelain and other botanical enzymes; subtilisin, epidermin, nisin, naringinase(L-rhammnosidase) urokinase and other bacterial enzymes. Said lipase include, but are not limited to, triacyl-glycerol lipases, monoacyl-glycerol lipases, lipoprotein lipases, e.g. steapsin, erepsin, pepsin, other mammalian, botanical, bacterial lipases and purified ones. Natural papain is preferred as said enzyme. Further, stimulating hormones, e.g. insulin, can be used together with these enzymes to boost the effectiveness of them.

The active may also be one or more plant extract. Examples of these components are as follows: Ashitaba extract, avocado extract, hydrangea extract, Althea extract, Arnica extract, aloe extract, apricot extract, apricot kernel extract, Ginkgo Biloba extract, fennel extract, turmeric[Curcuma] extract, oolong tea extract, rose fruit extract, Echinacea extract, Scutellaria root extract, Phellodendro bark extract, Japanese Coptis extract, Barley extract, Hyperium extract, White Nettle extract, Watercress extract, Orange extract, Dehydrated saltwater, seaweed extract, hydrolyzed elastin, hydrolyzed wheat powder, hydrolyzed silk, Chamomile extract, Carrot extract, Artemisia extract, Glycyrrhiza extract, hibiscustea extract, Pyracantha Fortuneana Fruit extract, Kiwi extract, Cinchona extract, cucumber extract, guanocine, Gardenia extract, Sasa Albo-marginata extract, Sophora root extract, Walnut extract, Grapefruit extract, Clematis extract, Chlorella extract, mulberry extract, Gentiana extract, black tea extract, yeast extract, burdock extract, rice bran ferment extract, rice germ oil, comfrey extract, collagen, cowberry extract, Gardenia extract, Asiasarum Root extract, Family of Bupleurum extract, umbilical cord extract, Salvia extract, Saponaria extract, Bamboo extract, Crataegus fruit extract, Zanthoxylum fruit extract, shiitake extract, Rehmannia root extract, gromwell extract, Perilla extract, linden extract, Filipendula extract, peony extract, Calamus Root extract, white birch extract, Horsetail extract, Hedera Helix(Ivy) extract, hawthorn extract, Sambucus nigra extract, Achillea millefolium extract, Mentha piperita extract, sage extract, mallow extract, Cnidium officinale Root extract, Japanese green gentian extract, soybean extract, jujube extract, thyme extract, tea extract, clove extract, Gramineae imperata cyrillo extract, Citrus unshiu peel extract Japanese Angellica Root extract, Calendula extract, Peach Kernel extract, Bitter orange peel extract, Houttuyna cordata extract, tomato extract, natto extract, Ginseng extract, Green tea extract (camelliea sinesis), garlic extract, wild rose extract, hibiscus extract, Ophiopogon tuber extarct, Nelumbo nucifera extract, parsley extract, honey, hamamelis extract, Parietaria extract, Isodonis herba extract, bisabolol extract, Loquat extract, coltsfoot extract, butterbur extract, Porid cocos wolf extract, extract of butcher's broom, grape extract, propolis extract, luffa extract, safflower extract, peppermintextract, linden tree extract, Paeonia extract, hop extract, pine tree extract, horse chestnut extract, Mizu-bashou [Lysichiton camtschatcese]extract, Mukurossi peel extract, Melissa extract, peach extract, cornflower extract, eucalyptus extract, saxifrage extract, citron extract, coix extract, mugwort extract, lavender extract, apple extract, lettuce extract, lemon extract, Chinese milk vetch extract, rose extract, rosemary extract, Roman Chamomile extract, and royal jelly extract.

### Antiparasite Agents

The biologically active substance contained in a composition of the present invention in a therapeutically effective amount may be an antiparasite agent, such as, but not limited to, hexachlorobenzene, carbamate, naturally occurring pyrethroids, permethrin, allethrin, malathion, piperonyl butoxide or mixtures of these drugs.

### Antimicrobial Agents

Antimicrobial agents, also referred to as germicidal agents, which may be used in compositions of the present invention include phenols, including cresols and resorcinols. Antibacterial compositions according to the present invention may be used to treat infections of the skin. An example of a very common skin infection is acne, which involve infestation of the sebaceous gland with p. acnes, as well as Staphylococus aurus or Pseudomonas. Various antibacterial agents have been utilized to treat acne, however, their efficacy is limited due to their low penetration into the hydrophobic environment of the sebaceous gland. The composition of the present invention, being hydrophobic by nature would facilitate an enhanced rate of penetration. Examples of useful antiacne actives include the keratolytics such as salicylic acid (o-hydroxybenzoic acid), derivatives of salicylic acid such as 5-octanoyl salicylic acid, and resorcinol; retinoids such as retinoic acid and its derivatives (e.g., cis and trans); sulfur-containing D and L amino acids and their derivatives and salts, particularly their N-acetyl derivatives, a preferred example of which is N-acetyl-L-cysteine; lipoic acid; antibiotics and antimicrobials such as benzoyl peroxide, octopirox, tetracycline, 2,4,4′-trichloro-2′-hydroxy diphenyl ether, 3,4,4′-trichlorobanilide, azelaic acid and its derivatives, phenoxyethanol, phenoxypropanol, phenoxyisopropanol, ethyl acetate, clindamycin and meclocycline; sebostats such as flavonoids; and bile salts such as scymnol sulfate and its derivatives, deoxycholate and cholate.

Another example is parachlorometaxylenol, which is an antimicrobial agent and is suitable for use in the compositions described in the present invention.

Phenols, in concentrations of about 0.2, 1.0, and 1.3 percent by weight are bacteriostatic, bactericidal, and fungicidal, respectively. Several phenol derivatives are more potent than phenol itself, and the most important among these are the halogenated phenols and bis-phenols, the alkyl-substituted phenols and the resorcinols.

Hydrophobic antibacterials useful in the present invention include triclosan, triclocarbon, eucalyptol, menthol, methylsalicylate, thymol, and mixtures thereof. Preferred are triclosan and triclocarbon.

### Antifungal Agents

Fungal infections are another object of treatment using the compositions of the present invention. Superficial fungal infection of the skin is one of the commonest skin disease seen in general practice. Dermatophytosis is probably the most common superficial fungal infection of the skin. It is caused by a group of fungi, which are capable of metabolizing the keratin of human epidermis, nails or hair. There are 3 genera of dermatophytes causing dermatophytosis i.e., microsporum, trichophyton and epidermophyton.

Candidiasis is an infection caused by the yeast like fungus Candida albicans or occasionally other species of Candida. Clinical syndromes of candidiasis include (a) oral candidiasis (oral thrush); (b) candidiasis of the skin and genital mucous membrane; and (c) candida paronychia, which inflicts the nail.

The composition of the present invention can contain an antifungal drug, which is active against dermatophytes and candida. The drug may include azoles, diazoles, triazoles, miconazole, fluconazole, ketoconazole, clotrimazole, itraconazole griseofulvin, ciclopirox, amorolfine, terbinafine, Amphotericin B, potassium iodide, flucytosine (5FC) and any combination thereof at a therapeutically effective concentration. U.S. Pat. No. 4,352,808 discloses 3-aralkyloxy-2,3-dihydro-2-(1H-imidazolylmethyl)benzo[b]thiophene compounds having antifungal and antibacterial activity.

### Steroidal Antiinflammatory Agents

Suitable steroidal antiinflammatory agents usable in the composition of the present invention may include, although are not limited to, corticosteroids such as hydrocortisone, hydroxyltriamcinolone alphamethyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclarolone acetonide, fludrocortisone, flumethasone pivalate, fluosinolone acetonide, fluocinonide, flucortine butylester, fluocortolone, fluprednidene (fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenalone acetonide, medrysone, amc, amcinafide, betamethasone and the balance of its esters, chlorprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylproprionate, hydrocortamate, meprednisone, paramethasone, prednisolone, prednisone, beclomethasone dipropionate, betamethasone dipropionate, triamcinolone, and mixtures thereof may be used. The preferred steroidal antiinflammatory for use in the present invention is hydrocortisone.

Psoriasis is a very common chronic inflammatory skin disease, which may be the target of treatment using a composition of the present invention. Psoriasis is marked by periodic flare-ups of sharply defined red patches covered by a silvery, flaky surface.

Corticosteroid ointments, greasy preparations containing small amount of water, are commonly used for treating psoriasis. Their main disadvantage is in their stickiness, which remains for long time after treatment is over. Examples of other inflammatory diseases or disorders, which can be treated by the composition of the present invention, wherein the drug is a steroid are: seborrheic dermatitis of the face and trunk, seborrheic blepharitis, contact dermatitis, stasis dermatitis (gravitational eczema; varicose eczema), exfoliative dermatitis (erythroderma), lichen simplex chronicus, pemphigus, conjuctivitis and uveitis.

Topical antihistaminic preparations currently available include 1 percent and 2 percent diphenhydramine (Benadryl® and Caladryl®), 5 percent doxepin (Zonalon®) cream, phrilamine maleate, chlorpheniramine and tripelennamine, phenothiazines, promethazine hydrochloride (Phenergan®) and dimethindene maleate. These drugs, as well as additional antihistamines can also be included in the composition of the present invention.

Additionally, so-called “natural” antiinflammatory agents are useful in context of the present invention. For example, candelilla wax, alpha bisabolol, aloe vera, Manjistha (extracted from plants in the genus Rubia, particularly Rubia cordifolia), and Guggal (extracted from plants in the genus Commiphora, particularly Commiphora mukul, may be used as an active ingredient in the composition of the present invention.

### Non-Steroidal Antiinflammatory Drugs (NSAIDs)

Another embodiment of the present invention is administration of non-steroidal antiinflammatory drugs (herein NSAIDs) using a composition of the present invention. NSAIDs have been used extensively in recent years for treatment of chronic rheumatic or arthritic conditions and for management of pain. The compounds are believed to bring relief by inhibiting biosynthesis of prostaglandins at affected joints or in other tissue areas.

Salicylic acid, or aspirin, and ibuprofen are well-known examples of NSAIDs drugs. Examples of NSAIDs include the following categories: propionic to acid derivatives; acetic acid derivatives; fenamic acid derivatives; biphenylcarboxylic acid derivatives; and oxicams. All of these NSAIDs are fully described in the U.S. Pat. No. 4,985,459 to Sunshine et al. which is incorporated herein by reference. Examples of useful NSAIDs include acetyl salicylic acid, ibuprofen, naproxen, benoxaprofen, flurbiprofen, fenoprofen, fenbufen, ketoprofen, indoprofen, pirprofen, carprofen, oxaprozin, pranoprofen, mniroprofen, tioxaprofen, suprofen, alminoprofen, tiaprofenic acid, fluprofen and bucloxic acid.

### Antioxidants/Radical Scavengers

Suitable antioxidants/radical scavengers useful in context of the present invention include ascorbic acid (vitamin C) and its salts, tocopherol (vitamin E), and its derivatives such as tocopherol sorbate, other esters of tocopherol, butylated hydroxy benzoic acids and their salts, 6-hydroxy-2,5,7,8-tetramethylchroman-2-carboxylic acid (commercially available under the trade name Trolox®), gallic acid and its alkyl esters, especially propyl gallate, uric acid and its salts and alkyl esters, sorbic acid and its salts, the ascorbyl esters of fatty acids, amines (e.g., N,N-diethylhydroxylamine, amino-guanidine), sulfhydryl compounds (e.g., glutathione), and dihydroxy fumaric acid and its salts may be used, as well as EDTA, BHT and the like.

### Antibiotics

Antibiotics which may be used in context of the composition of the present invention, include, but are not limited to, chloramphenicol, tetracyclines, synthetic and semi-synthesic penicillins, beta-lactames, quinolones, fluoroquinolnes, macrolide antibiotics, peptide antibiotics, cyclosporines, erytromycin and clinndamycin.

### Topical Anesthetics

Examples of topical anesthetic drugs useful in context of the composition of the present invention include benzocaine, lidocaine, bupivacaine, chlorprocaine, dibucaine, etidocaine, mepivacaine, tetracaine, dyclonine, hexylcaine, procaine, cocaine, ketamine, pramoxine, phenol, and pharmaceutically acceptable salts thereof.

### Retinol

Another preferred group of drugs useful in context of the composition of the present invention include retinol, all trans retinoic acid and derivatives, isomers and analogs thereof, collectively termed “retinoids”. Compositions according to the present invention, which contain retinoids as the active ingredient can be used for the treatment of acne, seborrea, various dermatoses, inflammation of the skin, mucosal membranes, eye, vagina and the rectum, psoriasis and cancers, by application onto the affected area.

### Anti-Viral Agents

Any anti-viral agent well-known to one of skill in the art can be used in the compositions and the methods of the invention. Non-limiting examples of anti-viral agents include proteins, polypeptides, peptides, fusion protein antibodies, nucleic acid molecules, organic molecules, inorganic molecules, and small molecules that inhibit or reduce the attachment of a virus to its receptor, the internalization of a virus into a cell, the replication of a virus, or release of virus from a cell. In particular, anti-viral agents include, but are not limited to, nucleoside analogs (e.g., zidovudine, acyclovir, acyclovir prodrugs, famciclovir, gangcyclovir, vidarabine, idoxuridine, trifluridine, and ribavirin), n-docosanoll foscarnet, amantadine, rimantadine, saquinavir, indinavir, ritonavir, idoxuridine. alpha-interferons and other interferons, and AZT.

### Anti-Cancer Drugs

Examples of anti-cancer agents include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bisphosphonates (e.g., pamidronate (Aredria), sodium clondronate (Bonefos), zoledronic acid (Zometa), alendronate (Fosamax), etidronate, ibandornate, cimadronate, risedromate, and tiludromate); bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin-2 (including recombinant interleukin 2, or rIL2), interferon alpha-2a; interferon alpha-2b; interferon alpha-n1; interferon alpha-n3; interferon beta-I a; interferon gamma-I b; iproplatin; irinotecan hydrochloride; lanreotide acetate; letrozole; leuprolide acetate; liarozole hydrochloride; lometrexol sodium; lomustine; los oxantrone hydrochloride; masoprocol; maytansine; mechlorethamine hydrochloride; anti-CD2 antibodies; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epothilone A; epothilone B; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; HMG CoA reductase inhibitors (e.g., atorvastatin, cerivastatin, fluvastatin, lescol, lupitor, lovastatin, rosuvastatin, and simvastatin); hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; LFA-3TIP (Biogen, Cambridge, Mass.; U.S. Pat. No. 6,162,432); liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MWF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl-lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RH retinamide; rogletimide; rohitukine; romurtide; roquinimex; rubiginone B 1; ruboxyl; safingol; saintopin; SarCNU; sarcophytol A; sargramostim; Sd±1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; 5-fluorouracil; leucovorin; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; thalidomide; velaresol; veramine; verdins; verteporfin; vinorelbine; vinxaltine; vorozole; zanoterone; zeniplatin; zilascorb; and zinostatin stimalamer.

### Other Drugs

A broad range of analgesics may be utilized including, without limitation, morphine, codeine, heroine, methadone, thebaine, orpiarine, buprenorphine, morphinans, benzomorphans, acetaminophen, butorphanol, diflunisal, fenoprofen, fentanyl, fentanyl citrate, hydrocodone, aspirin, sodium salicylate, ibuprofen, oxymorphone, pentaxicine, naproxen, nalbuphine, mefenamic acid, meperidine and dihydroergotamine.

A typical narcotic antagonist is haloxone. Exemplary antitussive agents include, without limitation, diphenhydramine, guaifenesin, hydromorphone, ephedrine, phenylpropanolamine, theophylline, codeine, noscapine, levopropoxyphene, carbetapentane, chlorpehndianol and benzonatate.

Among the sedatives which may be utilized are, without limitation, chloral hydrate, butabarbital, alprazolam, amobarbital, chlordiazepoxide, diazepam, mephobarbital, secobarbital, diphenhydramine, ethinamate, flurazepam, halazepam, haloperidol, prochlorperazine, oxazepam, and talbutal.

Examples of cardiac drugs are, without limitation, quinidine, propranolol, nifedipine, procaine, dobutamine, digitoxin, phenyloin, sodium nitroprus side, nitroglycerin, verapamil HCl, digoxin, nicardipine HCl, and isosorbide dinitrate.

Antiemetics are illustrated by, without limitation, thiethylperazine, metoclopramide, cyclizine, meclizine, prochlorperazine, doxylamine succinate, promethazine, triflupromazine, and hydroxyzine.

A typical dopamine receptor agonist is bromocriptine mesylate. Exemplary amino acid, peptide and protein hormones include, without limitation, thyroxine, growth hormone (GH), interstitial cell stimulating hormone (ICSH), follicle-stimulating hormone (FSH), thyrotropic hormone (TSH), adrenocorticotropic hormone (ACTH), gonadotropin releasing hormone (GnRH) such as leuprolide acetate, vasopressin and their active degradation products Some products may have sufficiently high molecular weights that absorption through the stratum corneum or mucous membranes may be difficult. Therefore, the invention is applicable only to those hormones which have molecular weights and stereo configurations which will allow passage through the skin.

Female sex hormones which can be used include, without limitations, estradiol, diethylstilbestrol, conjugated estrogens, estrone, norethindrone, medroxyprogesterone, progesterone, and norgestrel.

Typical male sex hormones which may be utilized may be represented by, without limitation, testosterone, methyltestosterone, and fluoxymesterone.

iii) Optional Components—Enhancers

The present compositions may further contain additional optional components. In addition to the active agent and the silicone gum emulsions, various excipients and/or enhancing agents may be incorporated into the composition or topical formulations containing the present compositions. Throughout the application, the terms “excipient” and “enhancer” will be used interchangeably. As generally understood by those skilled in the art, excipients are additives that are used to convert the active agent into appropriate dosage forms that are suitable for application to the substrate. Excipients may also be added to stabilize the formulation and to optimize application characteristics.

The amount of optional component(s) iii) may vary, and is not limiting in the present compositons.

Examples of potential excipients include, but are not limited to, excipients that are found in the CTFA ingredient Database and the handbook of pharmaceutical excipients such as absorbents, anticaking agents, antioxidants (such as acetyl cysteine, arbutin, ascorbic acid, ascorbic acid polypeptide, ascorbyl dipalmitate, ascorbyl methylsilanol pectinate, ascorbyl palmitate, ascorbyl stearate, BHA, p-ydroxyanisole, BHT, t-butyl hydroquinone, caffeic acid, camellia sinensis oil, chitosan ascorbate, chitosan glycolate, chitosan salicylate, chlorogenic acids, cysteine, cysteine HCl, decyl mercaptomethylimidazole, erythorbic acid, diamylhydroquinone, di-t-butylhydroquinone, dicetyl thiodipropionate, dicyclopentadiene/t-butylcresol copolymer, digalloyl trioleate, dilauryl thiodipropionate, dimyristyl thiodipropionate, dioleyl tocopheryl methylsilanol, isoquercitrin, diosmine, disodium ascorbyl sulfate, disodium rutinyl disulfate, distearyl thiodipropionate, ditridecyl thiodipropionate, dodecyl gallate, ethyl ferulate, ferulic acid, hydroquinone, hydroxylamine HCI, hydroxylamine sulfate, isooctyl thioglycolate, kojic acid, madecassicoside, magnesium ascorbate, magnesium ascorbyl phosphate, melatonin, methoxy-PEG-7 rutinyl succinate, methylene di-t-butylcresol, methylsilanol ascorbate, nordihydroguaiaretic acid, octyl gallate, phenylthioglycolic acid, phloroglucinol, potassium ascorbyl tocopheryl phosphate, thiodiglycolamide, potassium sulfite, propyl gallate, rosmarinic acid, rutin, sodium ascorbate, sodium ascorbyl/cholesteryl phosphate, sodium bisulfite, sodium erythorbate, sodium metabisulfide, sodium sulfite, sodium thioglycolate, sorbityl furfural, tea tree (melaleuca aftemifolia) oil, tocopheryl acetate, tetrahexyldecyl ascorbate, tetrahydrodiferuloylmethane, tocopheryl linoleate/oleate, thiodiglycol, tocopheryl succinate, thiodiglycolic acid, thioglycolic acid, thiolactic acid, thiosalicylic acid, thiotaurine, retinol, tocophereth-5, tocophereth-10, tocophereth-12, tocophereth-18, tocophereth-50, tocopherol, tocophersolan, tocopheryl linoleate, tocopheryl nicotinate, tocoquinone, o-tolyl biguanide, tris(nonylphenyl) phosphite, ubiquinone, and zinc dibutyldithiocarbamate), antistatic agents, astringents, binders, buffering agents, bulking agents, chelating agents, colorants, cosmetic astringents, cosmetic biocides (such as aluminum phenolsulfonate, Ammonium Phenolsulfonate, Bakuchiol, Benzalkonium Bromide, Benzalkonium Cetyl Phosphate, Benzalkonium Chloride, Benzalkonium Saccharinate, Benzethonium Chloride, Potassium Phenoxide, Benzoxiquine, Benzoxonium Chloride, Bispyrithione, Boric Acid, Bromochlorophene, Camphor Benzalkonium Methosulfate, Captan, Cetalkonium Chloride, Cetearalkonium Bromide, Cetethyldimonium Bromide, Cetrimonium Bromide, Cetrimonium Chloride, Cetrimonium Methosulfate, Cetrimonium Saccharinate, Cetrimonium Tosylate, Cetylpyridinium Chloride, Chloramine T, Chlorhexidine, Chlorhexidine Diacetate, Chlorhexidine Digluconate, Chlorhexidine Dihydrochloride, p-Chloro-m-Cresol, Chlorophene, p-Chlorophenol, Chlorothymol, Chloroxylenol, Chlorphenesin, C₁₋clopirox Olamine, Climbazole, Cloflucarban, Clotrimazole, Coal Tar, Colloidal Sulfur, o-Cymen-5-ol, Dequalinium Acetate, Dequalinium Chloride, Dibromopropamidine Diisethionate, Dichlorobenzyl Alcohol, Dichlorophene, Dichlorophenyl Imidazoldioxolan, Dichloro-m-Xylenol, Diiodomethyltolylsulfone, Dimethylol Ethylene Thiourea, Diphenylmethyl piperazinylbenzimidazole, Domiphen Bromide, 7-Ethylbicyclooxazolidine, Fluorosalan, Formaldehyde, Glutaral, Hexachlorophene, Hexamidine, Hexamidine Diisethionate, Hexamidine Diparaben, Hexamidine Paraben, Hexetidine, Hydrogen Peroxide, Hydroxymethyl Dioxoazabicyclooctane, Ichthammol, Isopropyl Cresol, Lapyrium Chloride, Lauralkonium Bromide, Lauralkonium Chloride, Laurtrimonium Bromide, Laurtrimonium Chloride, Laurtrimonium Trichlorophenoxide, Lauryl Isoquinolinium Bromide, Lauryl Isoquinolinium Saccharinate, Laurylpyridinium Chloride, Mercuric Oxide, Methenamine, Methenammonium Chloride, Methylbenzethonium Chloride, Myristalkonium Chloride, Myristalkonium Saccharinate, Myrtrimonium Bromide, Nonoxynol-9 Iodine, Nonoxynol-12 Iodine, Olealkonium Chloride, Oxyquinoline, Oxyquinoline Benzoate, Oxyquinoline Sulfate, PEG-2 Coco-Benzonium Chloride, PEG-10 Coco-Benzonium Chloride, PEG-6 Undecylenate, PEG-8 Undecylenate, Phenol, o-Phenylphenol, Phenyl Salicylate, Piroctone Olamine, Sulfosuccinylundecylenate, Potassium o-Phenylphenate, Potassium Salicylate, Potassium Troclosene, Propionic Acid, PVP-Iodine, Quaternium-8, Quaternium-14, Quaternium-24, Sodium Phenolsulfonate, Sodium Phenoxide, Sodium o-Phenylphenate, Sodium Shale Oil Sulfonate, Sodium Usnate, Thiabendazole, 2,2′-Thiobis(4-Chlorophenol), Thiram, Triacetin, Triclocarban, Triclosan, Trioctyldodecyl Borate, Undecylenamidopropylamine Oxide, Undecyleneth-6, Undecylenic Acid, Zinc Acetate, Zinc Aspartate, Zinc Borate, Zinc Chloride, Zinc Citrate, Zinc Cysteinate, Zinc Dibutyldithiocarbamate, Zinc Gluconate, Zinc Glutamate, Zinc Lactate, Zinc Phenolsulfonate, Zinc Pyrithione, Zinc Sulfate, and Zinc Undecylenate), deodorant agents, emollients, external analgesics (such as Benzyl Alcohol, Capsicum Oleoresin (Capsicum Frutescens Oleoresin), Methyl Salicylate, Camphor, Phenol, Capsaicin, Juniper Tar (Juniperus Oxycedrus Tar), Phenolate Sodium (Sodium Phenoxide), Capsicum (Capsicum Frutescens), Menthol, Resorcinol, Methyl Nicotinate, and Turpentine Oil (Turpentine)), film formers, flavoring agents, fragrance ingredients, humectants, lytic agents, moisturizing agents, occlusivity enhancers, opacifying agents, oxidizing agents (such as Ammonium Persulfate, Calcium Peroxide, Hydrogen Peroxide, Magnesium Peroxide, Melamine Peroxide, Potassium Bromate, Potassium Caroate, Potassium Chlorate, Potassium Persulfate, Sodium Bromate, Sodium Carbonate Peroxide, Sodium Chlorate, Sodium Iodate, Sodium Perborate, Sodium Persulfate, Strontium Dioxide, Strontium Peroxide, Urea Peroxide, and Zinc Peroxide), reducing agents (such as Ammonium Bisufite, Ammonium Sulfite, Ammonium Thioglycolate, Ammonium Thiolactate, Cystemaine HCl, Cystein, Cysteine HCl, Ethanolamine Thioglycolate, Glutathione, Glyceryl Thioglycolate, Glyceryl Thioproprionate, Hydroquinone, p-Hydroxyanisole, Isooctyl Thioglycolate, Magnesium Thioglycolate, Mercaptopropionic Acid, Potassium Metabisulfite, Potassium Sulfite, Potassium Thioglycolate, Sodium Bisulfite, Sodium Hydrosulfite, Sodium Hydroxymethane Sulfonate, Sodium Metabisulfite, Sodium Sulfite, Sodium Thioglycolate, Strontium Thioglycolate, Superoxide Dismutase, Thioglycerin, Thioglycolic Acid, Thiolactic Acid, Thiosalicylic Acid, and Zinc Formaldehyde Sulfoxylate), penetration enhancers, pesticides, plasticizers, preservatives, skin bleaching agents such as hydroquinone, skin conditioning agents, skin protectants (such as Allantoin, Aluminum Acetate, Aluminum Hydroxide, Aluminum Sulfate, Calamine, Cocoa Butter, Cod Liver Oil, Colloidal Oatmeal, Dimethicone, Glycerin, Kaolin, Lanolin, Mineral Oil, Petrolatum, Shark Liver Oil, Sodium Bicarbonate, Talc, Witch Hazel, Zinc Acetate, Zinc Carbonate, and Zinc Oxide), slip modifiers, solubilizing agents, solvents, sunscreen agents (such as Aminobenzoic Acid, Cinoxate, Diethanolamine Methoxycinnamate, Digalloyl Trioleate, Dioxybenzone, Ethyl 4-[bis(Hydroxypropyl)]Aminobenzoate, Glyceryl Aminobenzoate, Homosalate, Lawsone with Dihydroxyacetone, Menthyl Anthranilate, Octocrylene, Octyl Methoxycinnamate, Octyl Salicylate, Oxybenzone, Padimate 0, Phenylbenzimidazole Sulfonic Acid, Red Petrolatum, Sulisobenzone, Titanium Dioxide, and Trolamine Salicylate), surface modifiers, surfactants and emulsifying agents, suspending agents, thickening agents, viscosity controlling agents including increasing or decreasing agents, UV light absorbing agent (such as Acetaminosalol, Allatoin PABA, Benzalphthalide, Benzophenone, Benzophenone 1-12, 3-Benzylidene Camphor, Benzylidenecamphor Hydrolyzed Collagen Sulfonamide, Benzylidene Camphor Sulfonic Acid, Benzyl Salicylate, Bornelone, Bumetriozole, Butyl Methoxydibenzoylmethane, Butyl PABA, Ceria/Silica, Ceria/Silica Talc, Cinoxate, DEA-Methoxycinnamate, Dibenzoxazol Naphthalene, Di-t-Butyl Hydroxybenzylidene Camphor, Digalloyl Trioleate, Diisopropyl Methyl Cinnamate, Dimethyl PABA Ethyl Cetearyldimonium Tosylate, Dioctyl Butamido Triazone, Diphenyl Carbomethoxy Acetoxy Naphthopyran, Disodium Bisethylphenyl Tiamminotriazine Stilbenedisulfonate, Disodium Distyrylbiphenyl Triaminotriazine Stilbenedisulfonate, Disodium Distyrylbiphenyl Disulfonate, Drometrizole, Drometrizole Trisiloxane, Ethyl Dihydroxypropyl PABA, Ethyl Diisopropylcinnamate, Ethyl Methoxycinnamate, Ethyl PABA, Ethyl Urocanate, Etrocrylene Ferulic Acid, Glyceryl Octanoate Dimethoxycinnamate, Glyceryl PABA, Glycol Salicylate, Homosalate, Isoamyl p-Methoxycinnamate, Isopropylbenzyl Salicylate, Isopropyl Dibenzolylmethane, Isopropyl Methoxycinnamate, Menthyl Anthranilate, Menthyl Salicylate, 4-Methylbenzylidene, Camphor, Octocrylene, Octrizole, Octyl Dimethyl PABA, Octyl Methoxycinnamate, Octyl Salicylate, Octyl Triazone, PABA, PEG-25 PABA, Pentyl Dimethyl PABA, Phenylbenzimidazole Sulfonic Acid, Polyacrylamidomethyl Benzylidene Camphor, Potassium Methoxycinnamate, Potassium Phenylbenzimidazole Sulfonate, Red Petrolatum, Sodium Phenylbenzimidazole Sulfonate, Sodium Urocanate, TEA-Phenylbenzimidazole Sulfonate, TEA-Salicylate, Terephthalylidene Dicamphor Sulfonic Acid, Titanium Dioxide, TriPABA Panthenol, Urocanic Acid, and VA/Crotonates/Methacryloxybenzophenone-1 Copolymer). Other possible excipients include, but are not limited to, sugars and derivatives (such as acacia, dextrin, dextrose, fructose, lactose, maltodextrin, mannitol, sorbitol, sucrose, and xylitol), starch derivatives, cellulosic materials (such as Na Carboxymethylcellulose, Microcrystalline Cellulose, Cellulose Acetate Phtalate, Na croscarmellose, methyl cellulose, Ethylcellulose, Hydroxyethylcellulose, Hydroxypropylcellulose, Hydroxypropylmethylcellulose, and Hydroxypropylmethyl cellulose phtalate), polysaccharides (such as dextrates, guar gum, and xanthan gum), polyether (such as poloxamer, and polyoxyethylene alkyl ethers), polyvinyl alcohols, acrylic and methacrylic acid polymers (such as Carbomer, Polacrilin potassium, and polymethacrylates), pyrrolidone derivatives (such as povidone, and crospovidone), glycuronam polymer and derivatives (such as alginic acid, alginate salts (Ca, Na)), solid diluents (such as salts of carbonate (Ca, Mg), Ca Phosphate derivatives, Ca Sulfate, Mg oxide, Potassium Chloride, Potassium citrate), solid lubricants (such as stearate derivatives (Ca, Mg), talc, zinc oxide), suspending agents (such as kaolin, Mg Al silicate, and carbon), cyclodextrins, and others (including Cholesterol, Fumaric acid, lecithin, gelatin, malic acid, Na bicarbonate, Na citrate salts, Na stearyl fumarate, Ti dioxide, and Zinc oxide).

Enhancers may also be exemplified by monohydric alcohols such as ethanol and isopropyl, butyl and benzyl alcohols, or dihydric alcohols such as ethylene glycol, diethylene glycol, or propylene glycol, dipropylene glycol and trimethylene glycol, or polyhydric alcohols such as butylene glycol, hexylene glycol, polypropylene glycol, ethylene glycol, and polyethylene glycol, which enhance drug solubility; polyethylene glycol ethers of aliphatic alcohols (such as cetyl, lauryl, oleyl and stearyl) including polyoxyethylene (4) lauryl ether, polyoxyethylene (2) oleyl ether and polyoxyethylene (10) oleyl ether commercially available under the trademark BRIJ® 30, 93 and 97, respectively, from Uniqema Americas LLC (Wilmington, Del.), and others such as BRIJ® 35, 52, 56, 58, 72, 76, 78, 92, 96, 700 and 721; vegetable, animal and fish fats and oils such as olive, canola, safflower, cod liver, and castor oils, squalene, lanolin; fatty acids such as oleic, linoleic, and capric acid, and the like; fatty acid esters such as propyl oleate, decyl oleate, isopropyl palmitate, glycol palmitate, glycol laurate, dodecyl myristate, isopropyl myristate and glycol stearate which enhance drug diffusibility; fatty acid alcohols such as oleyl alcohol and its derivatives; fatty acid amides such as oleamide and its derivatives; urea and urea derivatives such as allantoin which affect the ability of keratin to retain moisture; polar solvents such as dimethyldecylphosphoxide, methyloctylsulfoxide, dimethyllaurylamide, dodecylpyrrolidone, isosorbitol, dimethylacetonide, dimethylsulfoxide, decylmethylsulfoxide and dimethylformamide which affect keratin permeability; salicylic acid which softens the keratin; amino acids which are penetration assistants; benzyl nicotinate which is a hair follicle opener; and higher molecular weight aliphatic surfactants such as lauryl sulfate salts which change the surface state of the skin; and esters of sorbitol and sorbitol anhydride such as polysorbate 20 commercially available under the trademark Tween® 20 from Uniqema Americas LLC (Wilmington, Del.), as well as other polysorbates such as 21, 40, 60, 61, 65, 80, 81, and 85. Other enhancers include enzymes, ascorbic acid, panthenol, butylated hydroxytoluene, tocopherol, tocopherol acetate, tocopheryl linoleate, and other non-toxic enhancers commonly used in transdermal or transmucosal compositions.

Polyhydric alcohols also include glycols, triols and polyols having 4 to 6 alcoholic hydroxyl groups. Typical of said glycols are glycols containing 2 to 6 carbon atoms, e.g. ethylene glycol, propylene glycol, butylene glycol, polyethylene glycol (average molecular weight about 200-8,000, preferably about 200 to 6,000), etc. Examples of said triols include glycerin, trimethylolpropane, etc. Said polyols are exemplified by sorbitol (sorbit), polyvinylpyrrolidone, etc. These polyhydric alcohols may be used either singularly or in combination (preferably, of two or three). Thus, for example, glycerin or dipropylene glycol alone, or a mixture of either glycerin or dipropylene glycol with butylene glycol can be employed.

The present composition may be prepared by combining components i), ii), and optionally iii), and mixing. Mixing may be accomplished by simple stirring techniques, or alternatively may involve shear mixing. Any type of mixing and shearing equipment may be used to perform this step such as a batch mixer, planetary mixer, single or multiple screw extruder.

This disclosure further provides a method for delivering the healthcare active (as described above) to a substrate by applying a film of the present compositions containing the healthcare active to a substrate. Upon application, a film is formed on the substrate. Following application, the healthcare active is simultaneously delivered through the film to the substrate. In one embodiment, the substrate is human skin.

The method of delivering the active agent to the substrate further includes the step of applying the silicone emulsion to the substrate to deliver the active agent to the substrate. Upon application of the silicone emulsion, which contains the active agent, and upon exposure of the substrate to air, the water leaves the silicone gum emulsion and a film is formed on the substrate. The film contains the active agent. It is to be understood that exposing the substrate to air can essentially be instantaneous. There is no requirement that the substrate be exposed to air for prolonged periods of time. Instead, exposing the substrate to air can occur simultaneously as some of the silicone gum emulsion is being applied to the substrate.

In embodiments where the substrate is skin, the composition is applied to the skin, the composition may be applied, i.e., rubbed or coated, directly onto the skin. Alternatively, the composition may be deposited on a transdermal patch prior to application to the substrate.

### EXAMPLES

The following examples are included to demonstrate certain embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention. All percentages are in wt. %. All measurements were conducted at 23° C. unless indicated otherwise.

### Preparation of a Silicone Gum Emulsion

A representative silicone gum emulsion (SGE) was prepared as follows. Into a speed mixer cup, 35.02 g of silicone gum 36 (SGM 36, Dow Corning Corporation, Midland, Mich.) was weighed followed by the addition of 16.02 g of 3 mm solid glass beads (Fisher Scientific, Dubuque, Iowa) and 7.02 g of PLURONIC F 108 (BASF, Florham Park, N.J.). The cup was mixed in the speed mixer (Hauschild Type AM501 speed mixer, Hamm, Germany) for 2 minutes at 3500 rpm. The cup was taken out from the mixer and kept in the room temperature for about 5 minutes to cool down. The cup was mixed again for 1 minute. A small pin hole was made at the center of the lid. Into the same cup, 28 g of deionized water was added in 5 increments of 4, 4, 6, 6, and 8 g in each time. After each addition, the cup was mixed in the speed mixer for 30 seconds. An additional 30 seconds mixing was carried out at the end. The cup was kept in the room temperature for about 2 hours. The glass beads were settled at the bottom of the cup and were not used in the formulation preparation. The SGE obtained was used to make the active formulations as described below. The components of the SGE, water, PLURONIC F108, and SGM36 are mentioned individually in the formulation compositions (Tables).

### Examples 1-6

### Formulation Examples Using Ibuprofen

Formulation example 1 was prepared by weighing 0.2903 g of ibuprofen (IBP, USP grade, Spectrum chemical mfg. corp., New Brunswick, N.J.) in a speed mixer cup followed by the addition of 0.4594 g of propylene glycol (PG, USP/FCC grade, Fisher Scientific, Fair Lawn, N.J.) and 0.0510 g of oleic acid (OLAC, NF/FCC grade, Fisher Scientific, Fair Lawn, N.J.). The cup was closed with a lid and was gently mixed using vortex mixer until the IBP was completely dissolved or well dispersed. To this, 5.0087 g of the SGE was weighed into the cup, closed with lid and mixed in the speed mixer until a homogeneous material was obtained. The formulation material was mixed using a spatula in between the dental mixer mixing cycles to achieve the homogeneous formulation. Formulation examples 2-6 were prepared using a similar procedure as described above by changing the amount of individual components as mentioned in the Table 1. Ethyl acetate (EA, HPLC grade) and dimethylsulfoxide (DMSO, certified ACS grade) were supplied by ACROS (Fair Lawn, N.J.) and Fisher Scientific (Fair Lawn, N.J.) respectively. The SGE was added last in each formulation during the preparation.

TABLE 1
Composition of formulation examples 1-6.

| | Formulation examples | | | | | |
|---|---|---|---|---|---|---|
| | 1 | 2 | 3 | 4 | 5 | 6 |
| Ingredients | % (w/w) | | | | | |
| SGM 36 (from SGE) | 43.1 | 43.0 | 47.5 | 39.3 | 43.0 | 41.3 |
| PLURONIC F108 (from SGE) | 8.6 | 8.6 | 9.5 | 7.9 | 8.6 | 8.3 |
| Water (from SGE) | 34.5 | 34.4 | 38.0 | 31.5 | 34.4 | 33.1 |
| PG | 7.9 | — | — | 7.4 | — | 7.9 |
| OLAC | 0.9 | — | — | 0.8 | — | 0.9 |
| EA | — | 9.0 | — | 8.1 | — | — |
| DMSO | — | — | — | — | 9.0 | 3.6 |
| IBP | 5.0 | 5.0 | 5.0 | 5.0 | 5.0 | 5.0 |
| Total | 100.0 | 100.0 | 100.0 | 100.0 | 100.0 | 100.0 |

The permeability behavior, the flux (or the amount of IBP delivered through skin per unit area per unit time, (μg/cm²/hr)) of the IBP from the above formulations was determined using Franz cell permeability experiment set-up at 32° C. and using the epidermis from human cadaver skin. In the Franz cell set-up, initially the bottom compartment of a cell was placed in the unit and filled with 3 mL of phosphate buffered saline (PBS, pH 7.4). A small magnetic stir bar was added to the cell. The permeation area in the Franz cell was 0.63 cm². The thawed epidermis of skin membrane (as a circle, 1.5875 cm diameter, 1.98 cm² area) was carefully transferred to the opening of the bottom compartment. For each formulation, 3 cells (triplicate) were prepared. A known amount (20±1 mg for formulations 4, 5, 6 and bench mark; 10±1 mg for all other formulations and the corresponding bench mark) of the formulation was taken using positive displacement pipette, applied on the skin and spread manually to visibly as homogeneous as possible. The top compartment (cap) of the Franz cell was attached now on top of the skin and both the top and bottom compartments were clamped together. PBS was added to the right volume (˜5 mL) of the cell and now the permeability experiment was started. The experiment was carried out for 8 hours. During the 8 hour period, 1 mL of sample was collected from the bottom compartment and replaced with fresh PBS solution at 0.5, 1, 2, 4, 6 and 8 hours. The experiment was stopped after collecting the sample at 8 hour. All samples collected were taken for ultra performance liquid chromatography (UPLC) analysis to determine the IBP concentration. The commercially available bench mark product (Ibutop, 5% Ibuprofen topical gel, DOLORGIET, Bonn, Germany) was used in each set of permeability experiments carried out for the formulation examples.

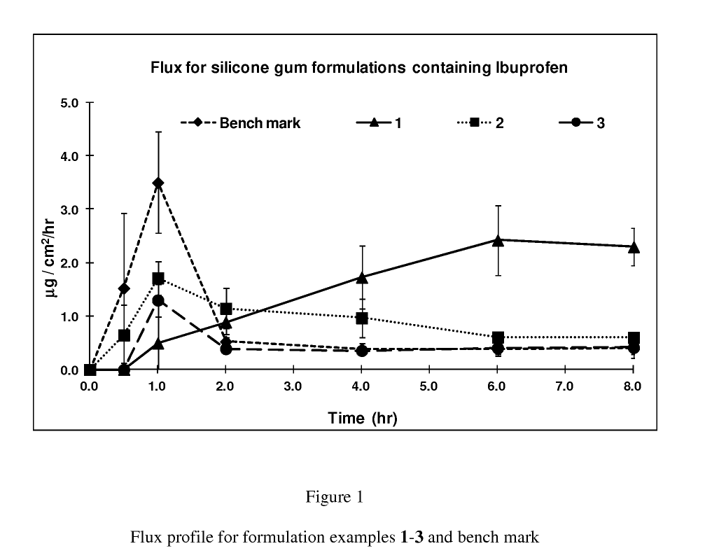

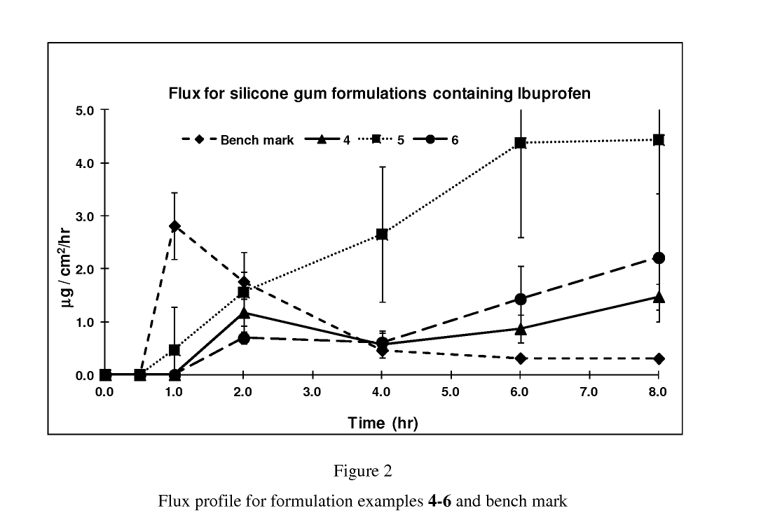

The flux profile for the formulation examples 1-3 and 4-6 are provided in the FIGS. 1 and 2. The figure also shows the flux profile for the bench mark. The flux experiment was carried out for each set of formulations and bench mark at the same time using the same skin epidermis.

### Examples 7-12

### Formulation Examples Using Diclofenac Sodium

Formulation example 7 was prepared by weighing 0.0564 g of diclofenac sodium (DCF, USP grade, Spectrum chemical mfg. corp., New Brunswick, N.J.) in a speed mixer cup followed by the addition of 0.4607 g of PG and 0.0512 g of OLAC. The cup was closed with a lid and was gently mixed using vortex mixer until the DCF was completely dissolved. To this, 5.0182 g of the SGE was weighed, closed with lid and mixed in the speed mixer until a homogeneous material was obtained. The formulation material was also mixed using a spatula in between the speed mixer mixing cycles. Formulation examples 8-12 were prepared using similar procedure described above by changing the amount of individual components as mentioned in the Table 2. The SGE was added last in each formulation during the preparation.

TABLE 2
Composition of formulation examples 7-12.

| | Formulation examples | | | | | |
|---|---|---|---|---|---|---|
| | 7 | 8 | 9 | 10 | 11 | 12 |
| Ingredients | % (w/w) | | | | | |
| SGM 36 (from SGE) | 44.9 | 42.9 | 49.5 | 41.1 | 39.3 | 37.4 |
| Pluronic F108 (from SGE) | 9.0 | 8.6 | 9.9 | 8.2 | 7.9 | 7.5 |
| Water (from SGE) | 35.9 | 34.3 | 39.6 | 32.9 | 31.4 | 30.0 |
| PG | 8.3 | 8.6 | — | 7.7 | 7.9 | 7.6 |
| OLAC | 0.9 | 0.9 | — | 0.9 | 0.9 | 0.8 |
| EA | — | — | — | 8.2 | 8.2 | 7.9 |
| DMSO | — | 3.7 | — | — | 3.4 | 7.8 |
| DCF | 1.0 | 1.0 | 1.0 | 1.0 | 1.0 | 1.0 |
| Total | 100.0 | 100.0 | 100.0 | 100.0 | 100.0 | 100.0 |

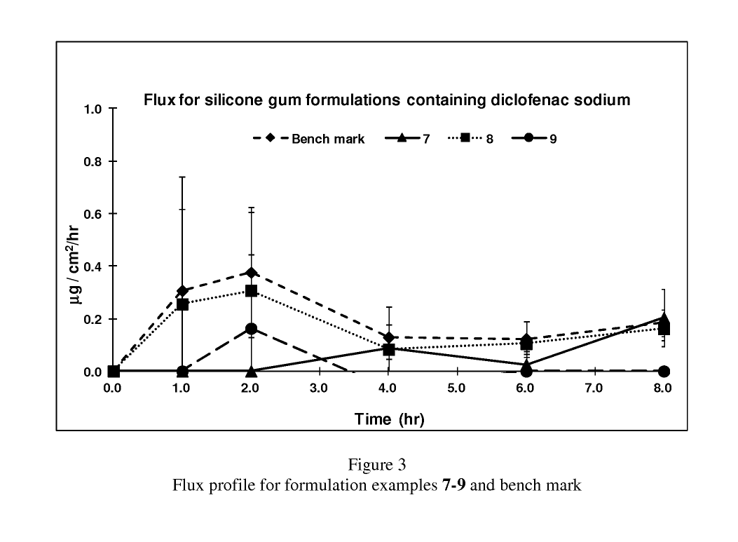

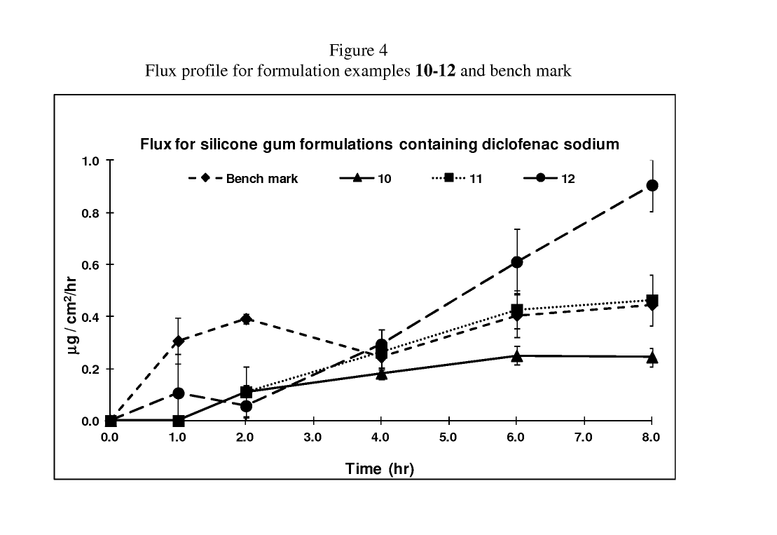

The permeability experiment was carried out using a Franz cell set-up as described above. The experiment was carried out for 8 hours. During the 8 hours period, 1 mL of sample was collected from the bottom compartment and replaced with fresh PBS solution at 1, 2, 4, 6 and 8 hours. The experiment was stopped after collecting the sample at 8 hour. All samples collected were taken for ultra performance liquid chromatography (UPLC) analysis to determine the DCF concentration. The commercially available bench mark product (Voltaren, diclofenac sodium topical gel 1%, Novartis, Parsippany, N.J.) was used in each set of permeability experiments carried out for the formulation examples. The flux profile for the formulation examples 7-9 and 10-12 are provided in the FIGS. 3 and 4. The figure also shows the flux profile for bench mark. The flux experiment was carried out for each set of formulations and bench mark at the same time using the same skin epidermis.

The representative formulations prepared using SGE delivered the drug through the skin and showed encouraging flux profiles. Formulation examples 1, 2, and 5 showed higher IBP delivery between 2 to 8 hours than that by bench mark. Formulations examples 4 and 6 showed higher IBP delivery between 4 to 8 hours compared to that for the bench mark. The bench mark showed an initial burst release (high drug delivery at the beginning) of IBP. This may be due to the presence of isopropyl alcohol (IPA). The initial burst release shown by the formulations was not equivalent to that shown by the bench mark. Attempts were made to incorporate lower aliphatic alcohols like IPA or ethanol (EtOH). However, both IPA and EtOH were not compatible with SGE and resulted emulsion breaking and/or coalescence of dispersed SGM. All the formulations were prepared using SGE with no modification of its preparation. However, the incorporation of IPA or EtOH might be possible in the SGE preparation itself. Moreover, formulations could be optimized using many other excipients other than PG, OLAC, EA, and DMSO to attain the initial burst release. DCF was also delivered by the SGE formulations and the flux profiles were encouraging. Formulation 8 showed very similar profile compared to that of the bench mark. Formulation 12 showed higher drug delivery between 4-8 hours compared to bench mark.

1. A composition comprising:
i) an aqueous silicone emulsion comprising;
A) 0.5 wt % to 95 wt % of a silicone gum, resin, or
pressure sensitive adhesive (PSA),
B) 0.1 to 90 wt % of an ethylene oxide/propylene oxide block copolymer,
and sufficient amount of water to sum all ingredients of the silicone gum emulsion to 100 weight percent,
ii) a healthcare active, and
iii) an optional enhancer(s).

2. The composition of claim 1 wherein the silicone emulsion is a water continuous emulsion.

3. The composition of claim 2 wherein the silicone emulsion is prepared by;
I) forming a dispersion of;
A) 100 parts of a silicone gum, resin, or
pressure sensitive adhesive (PSA),
B) 5 to 100 parts of a ethylene oxide/propylene oxide block copolymer,
II) admixing a sufficient amount of water to the dispersion from step I) to form an emulsion,
III) optionally, further shear mixing the emulsion.

4. The composition of claim 3 wherein the dispersion formed in step I) consists essentially of
A) 100 parts of a silicone gum, resin, or pressure sensitive adhesive (PSA),
B) 5 to 100 parts of an ethylene oxide/propylene oxide block copolymer.

5. The composition of claim 1 where the healthcare active is a non-steroidal anti-inflammatory drug.

6. The composition of claim 5 wherein the drug is ibuprofen or diclofenac.

7. The composition of claim 3, where the healthcare active is a non-steroidal anti-inflammatory drug.

8. The composition of claim 7, wherein the drug is ibuprofen or diclofenac.

9. The composition of claim 3, further comprising further shear mixing the emulsion.

10. The composition of claim 1, wherein the aqueous silicone emulsion comprises a silicone gum comprising a diorganopolysiloxane gum with a molecular weight sufficient to impart a William's plasticity number of at least about 30 or a hydroxyl terminated polydimethylsiloxane gum having a viscosity of at least 20 million cP at 25° C. at 0.01 Hz.

11. The composition of claim 1, wherein the aqueous silicone emulsion comprises a silicone resin comprising an MQ silicone resin or a silsesquioxane resin.

12. The composition of claim 1, wherein the aqueous silicone emulsion comprises a silicone PSA comprising the reaction product of a hydroxy endblocked polydimethylsiloxane polymer and a hydroxyl functional silicate or silicone resin.

13. The composition of claim 1, wherein the ethylene oxide/propylene oxide block copolymer comprises a surfactant having an HLB of at least 12.

14. The composition of claim 1, wherein the ethylene oxide/propylene oxide block copolymer comprises a poly(oxyethylene)-poly(oxypropylene)-poly(oxyethylene) tri-block copolymer.

15. The composition of claim 1, wherein the ethylene oxide/propylene oxide block copolymer comprises a tetrafunctional poly(oxyethylene)-poly(oxypropylene) block copolymer derived from the sequential addition of propylene oxide and ethylene oxide to ethylene diamine.

16. The composition of claim 1, wherein the aqueous silicone emulsion comprises 30 wt. to 60 wt. % of the silicone gum, resin, or PSA and 1 wt. % to 30 wt. % of the ethylene oxide/propylene oxide block copolymer.

17. A composition comprising:
i) an aqueous silicone emulsion comprising;
A) 0.5 wt % to 95 wt % of a silicone gum, resin, or
pressure sensitive adhesive (PSA),
B) 0.1 to 90 wt % of an ethylene oxide/propylene oxide block copolymer,
and sufficient amount of water to sum all ingredients of the silicone gum emulsion to 100 weight percent,
ii) a healthcare active, and
iii) an enhancer(s).

18. A method of preparing a silicone emulsion, the method comprising:
I) forming a dispersion of;
A) 100 parts of a silicone gum, resin, or PSA
B) 5 to 100 parts of an ethylene oxide/propylene oxide block copolymer,
II) admixing a sufficient amount of water to the dispersion from step I) to form an emulsion,
III) optionally, further shear mixing the emulsion

19. The method of claim 18, wherein the amount of water added in step II) is from 5 to 700 parts per 100 parts by weight of the dispersion from step I).

20. The method of claim 18, further comprising further shear mixing the emulsion.
